(19) United States
(12) Patent Application Publication
(10) Pub. No.: US 2005/0263149 A1
(43) Pub. Date: Dec. 1, 2005

(54) Aerosol drug delivery system employing formulation pre-heating

(76) Inventors: Peter D. Noymer, San Jose, CA (US); Justin D. Muratore, Berkeley, CA (US); Brooks M. Boyd, Berkeley, CA (US); David Cipolla, San Ramon, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/079,005

(22) Filed: Mar. 10, 2005

Related U.S. Application Data
(63) Continuation-in-part of application No. PCT/US03/29206, filed on Sep. 19, 2003.
Application No. PCT/US03/29206 is a continuation of application No. 10/251,898, filed on Sep. 19, 2002, now abandoned.

(51) Int. Cl. A61M 11/00; F23D 14/00

(52) U.S. Cl. 128/200.14; 128/203.26; 128/203.27; 128/204.17

(57) ABSTRACT

A formulation of a drug in a liquid carrier is heated in a controlled fashion and then aerosolized. The aerosolized formulation comprises particles having an aerodynamic diameter in a range of about 0.5 to about 12 micrometers. The particles are inhaled into the lungs of a human patient thereby delivering drug to the patient. By heating the formulation prior to aerosolization, the viscosity of the formulation is reduced thereby improving delivery efficiency. Repeatability of aerosol formation is improved by pre-heating the solution to the same or substantially the same temperature for successive delivery events.

### CROSS REFERENCES

This application is a continuation-in-part of International Patent Application Serial No. PCT/US2003/029206 filed Sep. 19, 2003 which claims priority to U.S. patent application Ser. No. 10/251,898 filed Sep. 19, 2002 (now abandoned) all of which are incorporated herein by reference in their entirety.

### FIELD OF THE INVENTION

The invention relates generally to devices and methods for generating medical aerosols and more particularly to devices and methods for pre-heating a liquid formulation comprising a drug to improve the repeatability and efficiency of aerosolized drug delivery.

### BACKGROUND

Intrapulmonary delivery of pharmaceutically active drugs is accomplished by a number of distinct methodologies. In accordance with one method, a pharmaceutically active drug is dispersed in a low boiling point propellant (a CFC or HFA) and loaded in a pressurized canister from which the drug/propellant formulation may be released by the use of a device generally known as a metered dose inhaler (MDI). Once released, the propellant evaporates and the patient inhales particles of the drug. Another method involves the use of a nebulizer. Nebulizers typically use vibration or jet nebulization to create a mist of fine particles from a solution or suspension of a drug. The mist is inhaled through the mouth and/or nose by the patient. In the case of the device shown in PCT publication WO/85/00112, mist is generated at a pair of orifices by way of a pumping system. The mist is carried by a stream of warmed air directed across the device from a heater to user interface/outlet ports. A reservoir containing medicant is remotely located from the airflow stream created by a blower. It is shown behind a wall portion of the device housing, separating it from a main chamber of the device. Heated air carrying the mist is maintained at a desired temperature to produce a combined effect of hyperthermic and microbicidal agent treatment for cold viruses and bacterias residing in the nasal passages of a user. In U.S. Pat. No. 5,461,695, another nebulizer is disclosed, in which a warmed aerosol is produced. Again, air for carrying the aerosolized material is heated alone.

In yet another method a dry powdered drug (which may be included in packets) is inhaled. These methods are hindered by significant problems relating to patient compliance and dosing as described further below.

The use of dry powders in systems presents some unique difficulties. Firstly, the dry powders are difficult to store and can be easily contaminated with water vapor causing the powders to clump together. Systems which do not include dry powders include the drug dissolved or suspended in a liquid carrier. Although there are advantages to these systems (e.g., avoiding the clumping of powder particles) these systems are also affected by moisture in the surrounding air, i.e. humidity. Specifically, such systems may use water as the carrier, i.e. a formulation comprised of a drug and water is used to create aerosolized particles. The carrier (such as the water) present in the particles may be evaporated after the particles are formed, either intentionally, or as a result of environmental factors.

However, the amount of evaporation varies depending on factors such as the environmental temperature and humidity. As taught in U.S. Pat. No. 5,957,124, others have taken action in order to reduce the effects of the surrounding temperature and humidity by heating the aerosolized particles. Further, various types of aerosol drug delivery systems have suggested the use of heating elements in order to heat the aerosol created as shown in the portable systems of U.S. Pat. No. 6,158,431.

The use of systems to heat the aerosol and thereby standardize particle size in different temperatures and humidities is particularly important when delivering drugs with a narrow therapeutic window such as insulin and monomeric insulin forms as disclosed in U.S. Pat. No. 5,970,973. Devices for heating aerosols can be applied to various different types of systems including nebulizer systems as disclosed in U.S. Pat. No. 4,911,157, humidifier systems as in U.S. Pat. No. 5,916,493 and electrospray systems as in U.S. Pat. No. 5,247,842.

However, these systems merely employ heating an aerosol after aerosolization. U.S. Pat. No. 5,957,124 does disclose a heating element that heats a formulation to be aerosolized, but only to facilitate the evaporation process. Likewise, as referenced in the '695 patent noted above, DE-A1-3043537 teaches a nebulizer in which the substance to be nebulized is warmed by an electric heating element. As characterized in the '695 patent, the purpose of such warming is to prevent the strong cooling effect caused by aerosolized flow when it comes into contact with the mucus membranes of a user.

In contrast to each of the approaches noted above, the present invention involves heating a liquid formulation prior to the creation of an aerosol for drug delivery in order to obtain features and advantages including reducing the formulation viscosity and/or improving the efficiency and repeatability of dosing. The present invention utilizes temperature control of formulation to be aerosolized in order to control its viscosity. Because the viscosity of many liquids behaves like water (varying greatly with temperature changes as might be expected in common operating conditions) absent temperature control according to the present invention, aerosolization of these liquids under differing ambient conditions may not be as efficient or repeatable as possible. By way of temperature-based formulation viscosity control, the present invention addresses such considerations.

### SUMMARY OF THE INVENTION

Systems for generating an aerosol from a liquid formulation are disclosed wherein the liquid formulation is heated prior to being aerosolized. The formulation is preferably comprised of a pharmaceutically active drug dissolved and/or suspended in a pharmaceutically acceptable carrier which may be, but is not limited to, water, ethanol or a mixture thereof.

The formulation is heated to reduce its viscosity and thereby improve the efficiency of aerosolization as well as the repeatability of dosing. As to the former consideration, reducing the viscosity of the formulation by heating allows for aerosolization of a higher percentage of the formulation as compared to an unheated, higher viscosity formulation. Still further, in many instances the formulation can be aerosolized using a lower input of energy, in the form of pressure work, ultrasonic excitation, air jet nebulization, and the like when heated to a desired temperature. Regarding repeatability of dosing, this consideration is served by heating the formulation to substantially the same temperature for subsequent delivery events. Taking such action removes system variables (e.g., due to changing environmental conditions) affecting aerosolization, thereby offering more consistent results.

The heater used in pre-heating the formulation according to the present invention may form part of the aerosolization device, be part of a separate formulation container or be provided by an altogether separate component. Whatever the case, the heater and/or aerosolization device may be powered by an energy supply such as a battery or battery pack held within the delivery device. Preferred variations of the invention provide such features in a portable package or set of components.

The present invention includes systems comprising any of the features described herein—alone or in combination with each other, to varying degrees. Methodology described in association with the apparatus disclosed also forms part of the invention. For example, an aspect of the invention involves aerosolized drug delivery wherein an aqueous formulation is heated, thereby reducing its viscosity, aerosolized, for example moved through a nozzle thereby creating an aerosol of small particles of formulation, and inhaled into the lungs of a patient, which is generally a human, for topical treatment of lung disease or to enter the patient's circulatory system for systemic effect.

The device, system and methodology of the invention provide for greater repeatability in dosing when delivering an aerosol to a patient. Although aerosolized delivery has a number of advantages one of the disadvantages has been the erratic nature of the dosing. The consistency in the dosing can be improved by heating the formulation within a defined range, obtaining a formulation viscosity within a defined range and obtaining particles within a defined range. The repeatability is increased by obtaining the same parameters each time the drug is delivered to the patient. Thus, if the same amount of energy is added and the formulation is heated to the same temperature and the same viscosity is obtained particle size will be the same with each dosing event which will further ensure repeatable dosing.

An aspect of the invention includes a method of drug delivery with an aerosol drug delivery system, said method comprising:

(a) providing a formulation at a first temperature wherein said formulation comprises a carrier selected from water ethanol;

(b) heating said formulation to a second, predetermined temperature to reach a desired formulation viscosity wherein said formulation second temperature is between about 5° C. and about 80° C.;

(c) forming an aerosol of at least a portion of said formulation after said heating.

Yet another aspect of the invention includes a method of the type described above wherein the formulation second temperature is between about 10° C. and about 60° C. and (a)-(c) are repeated a plurality of times and wherein said formulation second temperature is achieved within about ±5° C. each time (a)-(c) are repeated and wherein said formulation viscosity is controlled by said second, predetermined temperature within about ±2-% each time (a)-(c) are repeated.

Yet another aspect of the invention is a method of the type described above wherein the formulation second temperature is between about 20° C. and about 50° C. and (a)-(c) are repeated a plurality of times and wherein said formulation second temperature is achieved within about ±10° C. each time (a)-(c) are repeated and wherein said viscosity is controlled within about ±10% each time (a)-(c) are repeated.

Still yet another aspect of the invention is a method of the type described here wherein the formulation second temperature is achieved within about ±3° C. each time (a)-(c) are repeated and wherein said viscosity is controlled within about ±5% each time (a)-(c) are repeated and wherein between about 10 and about 200 millijoules per microliter is input to said formulation to perform said heating each time (b) is repeated.

As described here devices and systems of the invention are designed to carry out methods of the invention of the type described above.

### BRIEF DESCRIPTION OF THE DRAWINGS

The following figures diagrammatically illustrate aspects of the invention. For the sake of clarity, identical numerals indicate like components, where convenient. The invention is not limited to the variations pictured.

FIGS. 1A and 1B are perspective views of a formula container and pressurizing piston with a heater.

FIG. 2A is an exploded perspective view of the piston/heater in of FIGS. 1A and 1B in isolation; FIG. 2B is an assembled view that shown in FIG. 2A.

FIG. 3 is a schematic illustration of a delivery system employing a pressurizing piston with a heater.

FIG. 4 is a schematic illustration of a delivery system employing a formulation container with a heating element.

FIG. 5 is a schematic illustration of a delivery system employing a formulation heater separate from its pressurizing piston and formulation container.

### DETAILED DESCRIPTION

In describing the invention in greater detail than provided in the Summary and as informed by the Background above, basic methodology according to the present invention is described. This discussion is followed by discussion of suitable hardware for use in the invention. Methodology particularly associated with given hardware is discussed in parallel with the same.

Before the present invention is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “and,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs.

Definitions

The term “carrier” means a liquid, flowable, pharmaceutically acceptable excipient material, which a drug is suspended in, or more preferably dissolved in. Carriers used in the present invention typically do not adversely interact with the drug. Generally, they have properties which allow for the formation of aerosolized particles, preferably particles having an aerodynamic diameter in the range of about 0.5 to about 12.0 microns when a formulation comprising the carrier is forced through pores having a diameter of about 0.25 to about 6.0 microns. Preferred carriers include water, ethanol and mixtures thereof. Other carriers can be used provided that they can be formulated to create a suitable aerosol and do not adversely (or overly) affect the drug or human lung tissue.

The terms “formulation” and “liquid formulation” and the like are used interchangeably herein to describe any pharmaceutically active drug with a pharmaceutically acceptable carrier in flowable form having properties such that it can be aerosolized to particles having a diameter of about 0.5 to about 12.0 micrometers. Such formulations are preferably solutions (e.g., aqueous solutions, ethanolic solutions, aqueous/ethanolic solutions, saline solutions and colloidal suspensions). Formulations can be solutions or suspensions of a drug in a low liquid carrier, such as a boiling point propellant. Preferred formulations are drug(s) dissolved in water.

The term “aerosolization” means the atomization of a bulk liquid formulation into particles having an aerodynamic diameter ranging from about 0.5 to about 12.0 micrometers.

The term(s) “aerosol particles” means particles of formulation comprised of pharmaceutically active drug and carrier, which are formed upon aerosolization of the formulation.

Methodology

As shown in the following table, the viscosity of pure water varies significantly with temperature.

TABLE 1

| temp. (deg. C.) | viscosity (cS) |
|---|---|
| 2 | 1.70 |
| 7 | 1.45 |
| 12 | 1.25 |
| 17 | 1.10 |
| 22 | 0.97 |
| 27 | 0.87 |
| 37 | 0.70 |
| 47 | 0.58 |

see A. F. Mills, Heat and Mass Transfer, Richard D. Irwin, Inc., 1995, p. 1160.

The realities associated with such data (water being representative of such variation an other carries that may be employed in formulations) are addressed by the present invention. Namely, the invention provides methods for aerosolized drug delivery in which heating prior to aersolization of formulation is provided so that aersolization occurs at or about at the same carrier/formulation viscosity for each act of administration.

The method comprises heating a formulation comprising a pharmaceutically active agent and a liquid carrier using any suitable heating means/apparatus. Often, a metal heating element is employed. It may be provided with electrical energy from an internal power source such as a battery contained within the device. Still, other heating element and power supply configurations are possible within the scope of the present invention.

Prior to aerosolization by such means as elaborated upon below or otherwise, the formulation is heated to the desired temperature. The desired temperature may be any temperature between about 5° C. and about 80° C., but is preferably in a range of about 10° C. to about 60° C., and more preferably in the range of about 20° C. to about 50° C., most preferably above about 25° C. Heating to a temperature above room temperature or common ambient temperature(s) ensures aerosolization at the same or substantially the same temperature without need for maintaining the delivery device and/or the formulation to be delivered in a relatively cooler environment.

Preferably, the liquid formulation is set within a container that is monitored with a temperature detection device in connection with a monitoring system operatively coupled to the heating system so that the heater provides a sufficient amount of energy to heat the formulation to the desired temperature for each heating event. It is also possible to monitor or set the formulation temperature by monitoring the temperature of the heater employed and knowing a priori the relationship between the temperature of the formulation and that of the heater. A thermostat or temperature control element of any variety may be employed to monitor temperature however accomplished. Exemplary temperature sensors that may be used in the same include: thermocouples, thermistors, junction-based thermal sensors (e.g., diode or transistor temperature sensors), thermopiles, fiber optic detectors, acoustic temperature sensors, quartz and other resonant temperature sensors, thermo-mechanical temperature sensors and thin film resistive elements. Detailed discussion of many of these devices is presented in the “Micromachined Transducers Sourcebook,” by Gregory T. A. Kovacs, published by McGraw-Hill 1998. Other information regarding the sensors is well known in the art.

However configured, for optimal results it is important that the heating system be designed and controlled in a manner so as to heat the formulation to a temperature within a desired range of ± about 10° C., or preferably ± about 5° C., or more preferably ± about 3° C. In some instances, it may be desired to achieve even greater accuracy or proximity in temperature from one heating to the next or over a series of such events. It is also important not to over-heat the formulation, as many drugs are not stable at high temperatures.

Obtaining the same or substantially the same temperature (as within the temperature ranges given above) when heating the formulation is desirable so as to obtain a formulation that will have the same or substantially the same viscosity when the formulation is aerosolized. Thus, it is desirable for the formulation to have the same viscosity ± about 20%, or more preferably ± about 10% or even more preferably ± about 5% or less for each aerosolization event. By proper system design and execution of formulation heating, these results can be attained.

The energy input to the formulation in order to heat it may be a value between about 1 and about 1000 millijoules per microliter of formulation, or preferably in a range of 5 to 500 millijoules per microliter, or more preferably in the range of 10 to 200 millijoules per microliter. Aerosolization may result upon forcing formulation through at least one nozzle or orifice or a plurality of pores in a membrane or the like. Alternatively, the aerosol may be generated using electrohydrodynamic aerosol generation, jet nebulization, ultrasonic excitation, via at least one vibrating orifice plate, spinning top aerosol generation, or other methods/means of generating liquid aerosols. Examples of selected ones of these aerosolization means (alternately, atomization means) and associated methodology as may be employed in the present invention are provided in each of U.S. Pat. Nos. 6,014,970; 5,586,550; 5,758,637; 5,164,740; 6,235,177; 6,205,999; 6,085,740; 6,427,682; 5,938,117; 6,000,394; 5,957,389; 5,549,102; 5,461,695; 5,312,046; 6,397,838; 6,269,810; 5,511,726; 5,115,971; 4,261,512; 5,662,271; 5,497,944 and U.S. Patent Publication 2002/0026940, and such other references as cited herein that may be applicable, especially those noted below.

In employing any of these means or others as noted elsewhere, particularly below, it is to be appreciated that apparatus aspects of the present invention particularly concern hardware suited to deliver discrete doses, especially metered doses (in contrast to a continuous flow) of formulation. As such, at least with respect to devices according to the present invention, hardware not typically used for such purposes will be modified and/or collateral hardware particularly adapted for such use may be used in connection with the various possible aerosolization means. Still, the methodology of the present invention concerning the setting of formulation viscosity by controlling temperature may have broader applicability.

By way of example, nebulizers as may be used in connection with the present invention are disclosed in U.S. Pat. Nos. 5,226,411 and 5,259,370. The teachings of U.S. Pat. No. 5,855,564 may also be employed in the present invention. This patent discloses the use of a rotating cam to force formulations from collapsible containers that have porous membranes positioned thereon. When the formulation is forced through the membrane aerosolized particles are created having an aerodynamic diameter in a range of about 0.5 to about 12 micrometers, more preferably about 0.5 to about 6 micrometers. Other specific examples of delivery devices as may be employed in connection with the present invention are described in U.S. Pat. Nos. 5,522,385 and 5,957,124. In order to use any of these referenced systems in the present invention (modified or not), a heating element will be provided and be positioned so as to effectively heat the formulation employed prior to aerosolization by the means described.

When the formulation has a decreased viscosity as compared to the same formulation at a lower temperature, the formulation is more efficiently aerosolized, and in particular when forced under pressure through a porous membrane nozzle of the type described within U.S. Pat. No. 5,497,763. When a formulation which is not heated is aerosolized, the formulation is more viscous as compared to the heated formulation and the higher viscosity results in inefficiencies in the aerosolization process, often resulting in a non-aerosolized component of the liquid formulation being left in the device.

Whatever sort of device (including any the various aerosolization means noted above—modified to enable discrete dosing, or taken as-is) employed to aerosolize the formulation, the product of such action is inhaled by a patient into the lungs. Where smaller particles are concerned, deep penetration may be achieved, even to the alveolar level, thereby offering an effective drug administration pathway into a patient's bloodstream.

Regarding the formulation to be employed in such methodology as treated above, it may be such that the drug is completely dissolved in a solvent comprising water or ethanol or both. Alternatively, the drug may be suspended in the liquid as particles, preferably in the size range about 0.01 to about 20 micrometers or more preferably about 0.1 to about 4.0 micrometers.

When carrying out the methods of the invention, as mentioned above, it may be preferred that the heating system include a control or monitoring means so that the amount of energy supplied to the heating means allows for the heating of the formulation to the same or substantially the same temperature repeatedly. In this way, the methodology can be carried out without user regard for the surrounding environment. In other variations of the invention, instead of providing a monitoring system to control heating, the heater may be self-regulating. For instance, the heating means may comprise an electrical heating element that changes resistance in response to temperature, either in a gradual fashion, or in an essentially sudden fashion when a predetermined temperature is reached. This change in resistance will reduce the amount of energy delivered to the element, thus inhibiting further heating. The change in resistance can be a decrease in resistance as the temperature increases, or more preferably, an increase in resistance as temperature increases. Still further, heating may simply be controlled by selecting a desired amount of time to run the heater. In which case, the extent or duration of heating may be correlated to ambient or environmental conditions.

In order to carry out the methodology of the present invention, in an aersolization drug delivery system, one approach or another for pre-heating formulation to a desired temperature is provided. Thus, a patient using the device can activate the device and activate the heating means that then heats the formulation. The formulation is then aerosolized and the patient inhales the aerosol particles.

Each time the patient uses the device, the formulation is heated to the same or substantially the same temperature resulting in the formulation having the same or substantially the same viscosity and thereby resulting in aerosolization of the same or substantially the same amount of formulation that is inhaled by the patient. This aids in ensuring repeatability of dosing, which is particularly important when delivering drugs such as insulin or monomeric insulin that have a narrow therapeutic window.

Devices

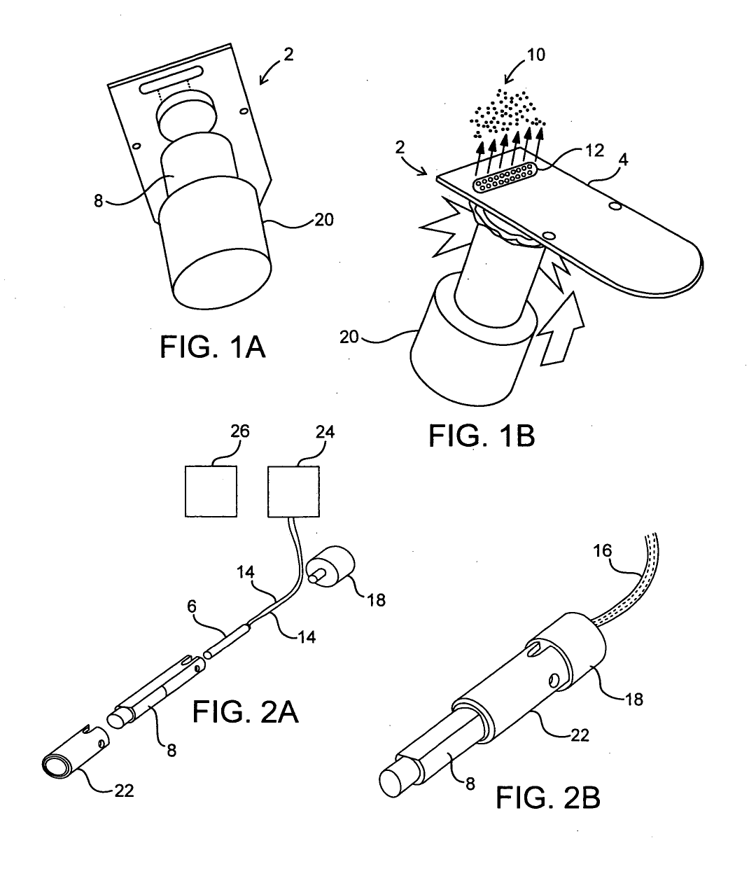

FIGS. 1A, 1B, 2A, 2B and 3-5 illustrate possible hardware aspects of the invention. Turning to FIGS. 1A-2B, a subsystem 2 for heating formulation (not shown) within a receptacle or container 4 (including an internal reservoir of the formulation) by a heating element 6 integral with the pressurizer/driver apparatus in the form of a piston 8 is shown. As the pressurization device or driver makes contact with the formulation container 4 to effect the pressurization of the contents therein, heating also takes place. Often, heating and pressurization will be effected in a two-stage process. First, the piston will be contacted with the container to transfer thermal energy. Next, when sufficient time has passed (or temperature information feed back information indicates a desired predetermined or selected temperature is reached) the piston completes its stroke to compress container or otherwise produce aersolized particles 10.

Formulation from within the container is aerosolized to from particles 10 by way of pores or orifice(s) 12 when container 4 is held in place while being pressurized by a piston 8 or other forcing means that is driven by a motive force (indicated by the large arrow in FIG. 1B). An air cylinder, crank, cam, or linkage, solenoid, piezo or motor driven device may be used to provide the driving force or itself serve as the driver to expel formulation from the container.

The approach to heating formulation in this variation of the invention is by integrating a heating element 6 with the piston, so that both heating and pressurization is achieved via contact with the formulation container. Thermal energy is directed at or toward the formulation by direct conduction through the wall of container 4.

One manner of achieving such integration is by providing a hollow piston 8 and inserting an electronic cartridge heater 6 therein. A suitable heater is manufactured by Omega Engineering, Inc. of Stamford, Conn. With this hardware, a spacer 18 is advantageously fitted the end of the piston to thermally isolate it from the driver device 20. Further, thermal isolation along the length of the piston may be provided by a secondary sleeve 22—which may also act as a linear bearing surface for the piston.

The heating element is energized by an energy source 24. As shown in FIG. 2A, electrical leads 14 may be provided for this purpose. As shown in FIG. 2B the leads may be bundled within a sheath 16 for ease of handling.

As noted above, the heater element may be controlled in a number of ways. One way is to provide temperature feedback data regarding the formulation in the container. A suitable chip and electronics may be used to direct such activity. With similar hardware, control may be provided in an open-loop fashion based on the amount of time the heater is energized. In a more basic system, the heating element may be controlled by selection of heater material properties such that the electrical resistance of the heating element increases gradually or suddenly at a pre-determined temperature level. Materials of this type are said to have a positive temperature coefficient, such as those manufactured by DBK—Heaters Engineering. As alluded to above, such control approaches may be used in any variation of the invention.

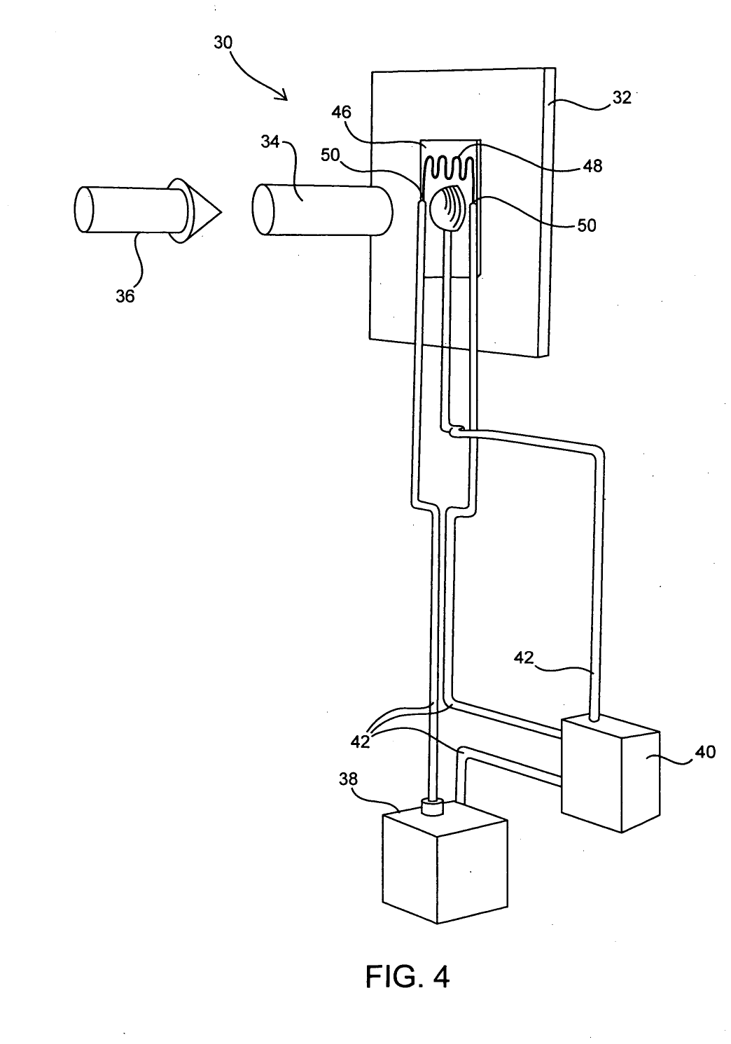

As in the other variations of the invention, formulation container 4 may comprise Kapton, a polyimide film manufactured by DuPont. Kapton is commonly used as an electrical insulator for thin-film heating elements, as in the “Kapton Insulated Flexible Heaters” manufactured by Omega Engineering, Inc. of Stamford, Conn. Therefore, integration of a heating element with the formulation container is feasible as shown in FIG. 4 with existing materials as may be used for similar containers as described in U.S. Pat. No. 5,497,763.

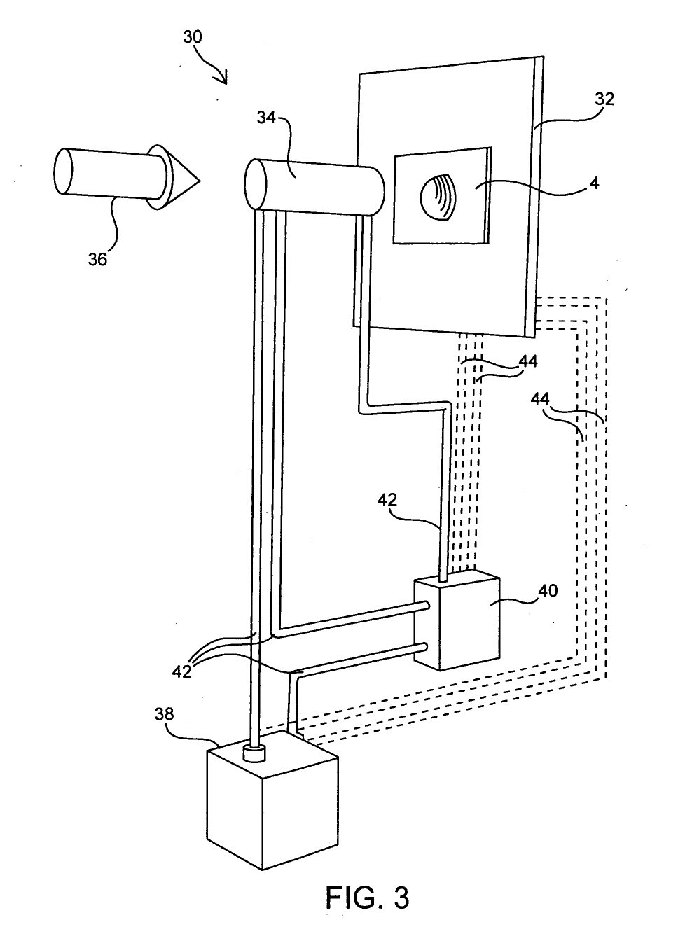

Regardless of such constructional details, FIG. 3 provides a schematic illustration of an overall delivery system 30 utilizing the approach taught in FIGS. 1A, 1B, 2A and 2B. During aerosolization, the formulation container 4 is held in place by a restraining means, such as the wall 32 of a delivery device while being pressurized by a piston or other pressurization means 34 that is driven by a source of motive force 36. As in the preceding figures, the approach for heating the formulation in this variation of the invention is by integrating a heating element with the pressurization means, so that as pressurization is achieved via contact, so is heating. The heating element would be energized by an energy source 38.

The heater/piston 34 is shown regulated by a controller 40 with the appropriate connections for energization and feedback 42. As indicated by the dashed connection lines 44, another option involves integrating a heating element into the surface or wall 32 opposing action by the motive force.

In FIG. 4, the container 46 of the formulation itself has an integral heating element 48, so that energizing the contacts to the container will heat the formulation even more directly. Based on exemplary power requirements for heating as expressed in Table 2 below, a 0.5″ radius, 5 W/in², Kapton-insulated flexible heater (e.g., Omega, KHR Series) may be used and attached externally to the formulation container. This heater would supply enough energy for a 14° C. temperature change in approximately 43 seconds, and could be easily powered by a portable battery pack. Of course, other heater types and heaters of differing capacities may be employed as well.

In integrating a heater into a formulation container 46, the combination may be designed to hold the heating element in the wall of the container or protruding from the wall into the formulation chamber defined by the walls of the container. By placing the heating element in the container walls or extending from the walls into the formulation it is possible to maximize the heating efficiency of the device and thereby minimize the amount of energy utilized from the power source such as an electrochemical cell or group of cells (i.e., a battery). The heating element can be electrically connected to the power source 38 and/or controller 40 via mating contacts 50 in the container and delivery device, respectively, coupled to lines 42.

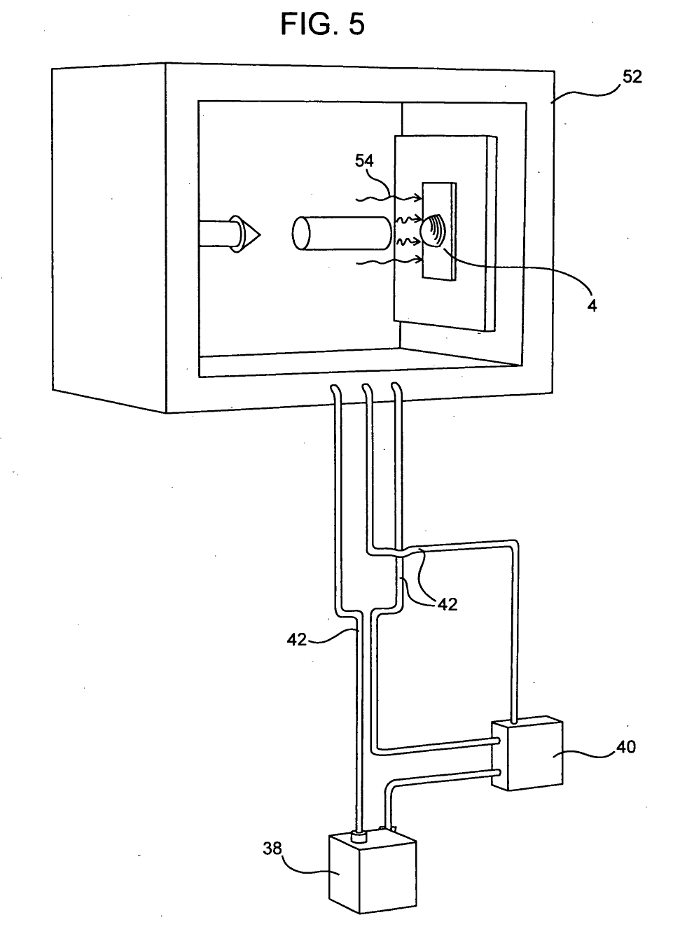

Another possibility for the invention is shown in FIG. 5. In this variation formulation heating is provided a separate sub-system 52 that encloses or surrounds some or all the formulation container 4. The aerosolization components (i.e. piston, 34, motivator 36, etc.) may be set within the heater housing 52 as shown or set near-by. For a portable delivery system, one area or compartment may be provided for formulation heating and another section, removed from the heater for later, for receiving the formulation container to aerosolize formulation therein. A heater provided in this manner (in which no direct contact with the container is made) may employ radiant coils (not shown), directing radiant energy and/or convective airflow 54 (such as produced by a fan (not shown) pointed toward the formulation container 4). Again, appropriate connections 42 may be provided to an energy source 38 and an optional controller 40.

Further, it is contemplated that an entire drug delivery system, or the formulation container itself, can be placed inside, in contact with or near to an auxiliary heater for pre-heating the formulation in the container prior to aersolization. In still another embodiment, the heat may be delivered to the formulation by placing the formulation and container in proximity to an element that is also heated for another purpose, such as an air heater that is used to heat the air and force evaporation of the aerosol. In such instances, however, an appropriate setup or control is required in order to be effective in accordance with the method parameters set forth above.

While variations of the invention may be more convenient that are easily applicable to using an internal power source, those employing auxiliary heaters may present certain advantages. One such advantage may involve the use of an external power source thereby improving the ability to heat the formulation without the need of using an internal power source of the device such as internal batteries. Still, it is contemplated that variations of the invention which integrate heater function into the container or other structure on-board the delivery device itself may include a power port or adapter to receive external power. In any case the invention is most particularly concerned with portable drug dispensing units. Such units are often characterized as weighting less than 1 kg, more preferably, less than 0.5 kg.

### EXAMPLES

Certain data is set forth below as exemplary of practice of the present invention. While efforts have been made to ensure accuracy with respect to numbers used and presented (e.g., amounts, temperature, etc.) some experimental errors and deviations may be expected. With respect to the information presented, temperatures are represented in degrees Centigrade, and pressure is at or near atmospheric.

In practicing an aspect of the present invention, table 2 shows an exemplary pre-heating data for 50 μL of an aqueous pharmaceutical solution formulation.

TABLE 2
Temperature rise of a 50-μL aqueous solution
as a function of power input and heating time

| power (W) | ΔT at 20 sec | ΔT at 30 sec |
|---|---|---|
| 0.4 | 2 | 3 |
| 0.9 | 5 | 8 |
| 2.1 | 14 | 19 |

As can be seen in Table 2, the amount of power and energy required from an energy source to warm a formulation from 5° C. (a typical cool environment) to 19° C. (a typical room-temperature environment) is rather small. Only 2.1 W are required for 20 seconds. Thus, approximately 42 Joules of energy are required, which can easily be supplied by a portable battery pack.

Reference to table 3 helps to illustrate the beneficial effect of heating formulation prior to aerosolization. The data in Table 3 represent aerosolization of a protein drug in aqueous solution at an ambient temperature of 5° C. The test data obtained reflects aerosolization efficiency as well as a measurement of delivery repeatability.

TABLE 3
Aerosolization efficiency and repeatability for an aqueous
protein formulation as a function of formulation temperature
and viscosity in a 5° C. environment.

| formulation | formulation | aerosolization | |
|---|---|---|---|
| temp. | viscosity | efficiency | relative std. |
| (deg. C.) | (cS) | (%) | deviation |
| 5 | 1.7 | 52 | 13% |
| 23 | 1.1 | 60 | 7% |
| 40 | 0.7 | 62 | 5% |

As a baseline, it may be observed that when the formulation temperature is the same as the ambient temperature, the aerosolization efficiency is slightly more than 50% and the variability of repeated aerosolization attempts is relatively large. However, when the formulation is heated to 23° C. with all else being the same (i.e., ambient temperature is at 5° C.), the aerosolization efficiency improves to roughly 60% and the variability of repeated aerosolizaton attempts is reduced from the previous case. When the formulation is heated to 40° C., both the efficiency and repeatability of aerosolization improve further.

Though the invention has been described in reference to certain examples, optionally incorporating various features, the invention is not to be limited to the set-ups described. The invention is not limited to the uses noted or by way of the exemplary description provided herein. It is to be understood that the breadth of the present invention is to be limited only by the literal or equitable scope of the following claims. That being said, we claim:

1. A method of drug delivery with an aerosol drug delivery system, said method comprising:
(a) providing a formulation at a first temperature wherein said formulation comprises a carrier selected from water ethanol;
(b) heating said formulation to a second, predetermined temperature to reach a desired formulation viscosity wherein said formulation second temperature is between about 5° C. and about 80° C.;
(c) forming an aerosol of at least a portion of said formulation after said heating.

2. The method of claim 1, wherein said formulation second temperature is between about 10° C. and about 60° C. and (a)-(c) are repeated a plurality of times and wherein said formulation second temperature is achieved within about ±5° C. each time (a)-(c) are repeated and wherein said formulation viscosity is controlled by said second, predetermined temperature within about ±2% each time (a)-(c) are repeated.

3. The method of claim 1, wherein said formulation second temperature is between about 20° C. and about 50° C. and (a)-(c) are repeated a plurality of times and wherein said formulation second temperature is achieved within about ±10° C. each time (a)-(c) are repeated and wherein said viscosity is controlled within about ±10% each time (a)-(c) are repeated.

4. The method of claim 3, wherein said formulation second temperature is achieved within about ±3° C. each time (a)-(c) are repeated and wherein said viscosity is controlled within about ±5% each time (a)-(c) are repeated and wherein between about 10 and about 200 millijoules per microliter is input to said formulation to perform said heating each time (b) is repeated.

5. The method of claim 2, wherein between about 1 and about 1000 millijoules per microliter is input to said formulation to perform said heating each time (b) is repeated.

6. The method of claim 3, wherein between about 5 and about 500 millijoules per microliter is input to said formulation to perform said heating each time (b) is repeated.

7. The method of claim 1, wherein said formulation comprises aerosolized particles including drug in suspension in liquid.

8. The method of claim 7, wherein said particles are about 0.01 to about 20 micrometers in diameter.

9. The method of claim 8, wherein said particles are about 0.1 to about 4 micrometers in diameter.

10. The method of claim 1, further comprising:
repeating said heating (b) of said formulation and said forming of aerosol (c), wherein said second, predetermined temperature for each repetition of (b) is substantially constant between each group of steps (a)-(c).

11. The method of claim 10, wherein said forming of aerosol (c) is performed at a substantially constant viscosity for each repetition of the steps (a)-(c).

12. The method of claim 1, wherein said forming of aerosol (c) is preformed by forcing said formulation through a nozzle.

13. The method of claim 1, wherein the aerosol comprises aerosolized particles having an aerodynamic diameter between about 0.5 micrometer and about 12 micrometers.

14. The method of claim 1, wherein said heating (b) is provided by an electrical heating element and thermal energy from said heating element is directly conducted to said formulation.

15. The method of claim 1, wherein heating (b) is controlled by a temperature monitoring and feedback system.

16. An aerosol drug delivery system comprising:
a drug delivery device housing,
a formulation container adapted to be received within said drug delivery device, a reservoir in said container being in fluid communication with an aerosolization means, wherein said aerosolization means is adapted to deliver discrete doses of formulation, and
a heater, said heater positioned to direct thermal energy at said container.

17. The system of claim 16, wherein thermal energy is conducted directly to said container wherein said thermal energy is chosen from radiant energy directed toward said container and convective energy directed toward said container and wherein said heater is provided integrally with said container.

18. The system of claim 17, wherein said aerosolization means comprises a piston and said heater is provided integrally with said piston further comprising an energy source to power said heater wherein said energy source comprises a battery and the system weights less than 0.5 kg.
